(19) United States
(12) Patent Application Publication
(10) Pub. No.: US 2010/0093674 A1
(43) Pub. Date: Apr. 15, 2010

(54) Skin cancer prevention method and product

(76) Inventor: John R. Person, Charlton, MA (US)

Correspondence Address: BLODGETT BLODGETT, 43 HIGHLAND STREET, WORCESTER, MA, 016092797, US

(21) Appl. No.: 11/196,946

(22) Filed: Aug. 4, 2005

(51) Int. Cl. A61K 31/593; A61P 35/00

(52) U.S. Cl. 514/167

(57) ABSTRACT

The increased use of sunscreens, while limiting damage to the DNA, may promote cancer growth by preventing vitamin D synthesis in the skin. According to the present invention, the beneficial effects of UV radiation are obtained by incorporating vitamin D into the topical after-sun lotions and creams, applied to the skin after exposure to the sun to prevent its harmful effects. Application of the after-sun product provides vitamin D, which is activated by the skin to calcitriol for cancer prevention. Because calcitriol also promotes cellular growth and differentiation, the topical after-sun product with vitamin D may be of benefit for photoaging.

### CROSS REFERENCES

This application claims priority to U.S. patent application Ser. No. 10/956,993, filed Sep. 29, 2004, and U.S. patent application Ser. No. 11/053,432, filed Feb. 8, 2005.

### BACKGROUND

1. Field of the Invention

The present invention relates to methods and products for preventing cancer through topical application of cancer inhibitors. More particularly, it relates to the use of after sun lotions and creams containing vitamin D₃, vitamin D derivatives (natural or synthetic), or cod-liver oil to inhibit skin cancers and other dermatological disorders related to ultraviolet light exposure.

2. Discussion of Related Art

It is common knowledge that we are experiencing an epidemic of skin cancer. Non-melanoma (basal and squamous cell carcinoma) skin cancer has increased by 3-8% per year since the 1960's, and the lifetime risk of malignant melanoma has increase from 1/500 in 1960 to 1/75 (estimated) in 2000. There is some evidence that vigorous use of sunscreens may reduce the incidence of nonmelanoma cancer. Sunscreens seek to block the ultraviolet light, which causes sunburn and is a probable cause of skin cancers. Sunscreens are rated with an sun-protective-factor (SPF) which is essentially a measure of protection against sunburn or ultraviolet B radiation. High SPF sunscreens have been available for almost 30 years and broader spectrum sunscreens, which also block some longwave ultraviolet light, for almost 15 years. There is some evidence that vigorous use of sunscreens may reduce the incidence of non-melanoma skin cancer, but it difficult to reconcile this with the non-melanoma skin cancer epidemic. The situation with melanoma is less clear. Some studies show a protective effect and others show that sunscreens may increase the risk of melanoma. There are no adequate animal models for basal cell carcinoma or melanoma So, testing of the effects of sunscreens with respect to skin cancer is difficult

Among the various hypotheses put forth to explain the failure of sunscreens, especially in the prevention of melanoma, is that sunscreens inhibit epidermal synthesis of vitamin D₃ (cholecalciferol), and that this promotes the growth of cancer. Calcitrol is a potent regulator of cell growth and differentiation. It also has an inhibitory effect on cellular death and new blood vessel growth, i.e., into tumors). Low vitamin D levels have been associated with breast, prostate, and colon cancer. New vitamin D analogues have been shown to be very effective in preventing chemical tumorgenesis in mice.

Vitamin D₃ is synthesized by epidermal keratinocytes on exposure to UVB, but must undergo activation first by 25-hydroxylation and then 1-alpha hydroxylation to convert it to 1,25-dihydroxyvitamin D₃, or calcitriol, the active form of vitamin D. Traditionally, these conversions have been thought to occur in the liver and kidney exclusively. Professor Michael F. Holick at Boston University has suggested that the UV blocking characteristics of sunscreens has inhibited the natural production of vitamin D₃ by the skin. He has created a controversy among the dermatological community by suggesting some unprotected exposure to natural or artificial sunlight for short periods of time in order to increase vitamin D production. Additionally, he has several patents, such as U.S. Pat. No. 5,422,099, relating to topical application of vitamin D precursors in conjunction with exposure to sunlight. The general consensus among dermatologists is that all UV radiation is problematic due to its connections with melanomas and other skin cancers. Furthermore, other sources of vitamin D, such as diet and supplements, can be used to provide a sufficient daily dose without production by the skin.

### SUMMARY OF THE INVENTION

The present invention provides a method of inhibiting skin cancers, precancers, photoaging and other dermatological disorders through use of vitamin D₃. According to one aspect of the invention, vitamin D₃ is provided as an ingredient in a topical lotion or cream associated with sun exposure. According to another aspect of then invention, cod-liver oil is provided as an ingredient in a topical lotion or cream associated with sun exposure as a source of vitamin D₃. A topical lotion or cream associated with sun exposure containing vitamin D₃ or cod-liver oil is applied to portions of the skin previously subjected to exposed to sunlight.

### DETAILED DESCRIPTION

According to traditional dogma, hepatic and renal activation of vitamin D₃ are necessary to produce calcitriol. However, it has been known for many years that the skin is capable of converting vitamin D₃ to calcitriol on its own, in addition to creating vitamin D₃ through exposure to sunlight. Bikle D D, Nemanic M K, Gee E, et al., 1,25 dihydroxyvitamin D₃ Production by Human Keratinocytes, Kinetics and Regulation, J Clin Invest 1986, 557-66; Bikle D D, Nemanic M K, Whitney J D et al., Neonatal Human Foreskin Keratinocytes Produce 1,25 dihydroxyvitamin D₃. Biochemistry 1986; 25:1545-8. Matsumoto K, Azuma Y, Kiyoki M, et al. Involvement of Endogenously Produced 1,25 dihydroxyvitamin D3 in the Growth and Differentiation of Human Keratinocytes, Biochim Biophys Acta 1997, 1092:311-8.

Recent studies have shown that melanoma patients have normal calcitriol serum levels, normal 25-hydroxyvitamin D₃ serum levels, and normal dietary vitamin D intake. Cornwell M L, et al., Prediagnositc Serum Levels of 1,25 dihydroxyvitamin D and Malignant Melanoma, Photodermatol Photoimmunol Photomed 1992, 9:109-12; Reichrath J, et al., No Evidence for Reduced 25-hydroxyvitamin D Serum levels in Melanoma Patients, Cancer Causes Control, 2004, 15:97; and Weinstock M A, et al., Case-Control Study of Melanoma and Dietary Vitamin D: Implications for Advocacy of Sun Protection and Sunscreen Use, J Invest Dermatol, 1992; 98:809-11. However, it is known that calcitriol inhibits the growth and invasion of melanoma cells and invasion. Colston K, et al., 1,25-Dihydroxyvitamin D₃ and Malignant Melanoma: The Presence of Receptors and Inhibition of Cell Growth in Culture, Endocrinology, 1981; 108:1083-6. Evans S R, et al., Vitamin D Receptor and Growth Inhibition by 1,25 dihydroxyvitamin D₃ in Human Malignant Melanoma Cell Lines, J Surg Res, 1996; 61:127-33. Yudon, K et al., 1 alpha, 25 Dihydroxyvitamin D₃ Inhibits In Vitro Invasiveness Through The Extracellular Matrix and In Vivo Pulmonary Metastasis of B16 Mouse Melanoma, J Lab. Clin Med 1999, 133:120-8. Furthermore, melanoma cells can express the vitamin D receptor and can activate vitamin D₃. Frankel, T L, et al., The Synthesis of Vitamin D Metabolites By Human Melanoma Cells, J Clin Endocrimol Metab, 1983, 57:627-631. Polymorphisms of the vitamin D receptor are also associated with an increased susceptibility to and worsened progress in melanoma. Halsall J A, et al., A novel Polymorphism in the IA Promoter Region of the Vitamin D Receptor Is Associated With Altered Susceptibility and Prognosis in Malignant Melanoma, Br J Cancer, 2004: 16:765-70. Hutchinson P E, et al., Vitamin D Receptor Polymorphisms Are Associated With Altered Prognosis in Patients With Melanoma, Clin Cancer Res, 2000; 6:498-504.

Thus, serum vitamin D levels and dietary intake appear to be of little importance in preventing melanomas despite the inhibiting effect of calcitriol. However, the ability of the epidermis to generate calcitriol, as suggested by the various studies, explains the conflicting data. Activation of vitamin D₃ to calcitriol within the epidermis puts the anti-tumor activity of calcitriol at the site of tumor formation and at the time of tumor formation in high concentration. As shown by K. Matsumoto, most of the calcitriol remains within the keratinocyte. This is also suggested by the fact that individuals who sunbathe do not experience elevated calcium levels (an obvious effect of both topical and circulating calcitriol). It also explains the increase in skin cancer despite widespread use of sunscreens. Exposure to UV radiation can increase the risk of cancer. On the other hand, it causes the skin to produce vitamin D which has a cancer inhibiting effect. The use of sunscreens prevents the formation of vitamin D in the skin. Accordingly, protection against sunburn seems not to be protection against skin cancer due to variations in vitamin D formation and activation.

The present invention resolves the conflict by providing another source of vitamin D to the skin in relation to exposure to the sun. Specifically, vitamin D₃ (cholecalciferol) or other biologically active vitamin D derivatives are added to topical applications of lotions or creams associated with sun exposure. U.S. patent application Ser. No. 11/053,432, entitled Skin Cancer Prevention Method and Product, by the present inventor, relates to the addition of vitamin D₃ and vitamin D derivatives to sunscreens. When the sunscreen is applied, it inhibits the UV radiation and its cancer causing effects. On the other hand, the vitamin D₃ is provided to the skin. The skin can activate the vitamin D₃ to calcitriol to provide the anti-cancer effects at the epidermis. The present invention relates to other applications of vitamin D₃ or its derivatives. Specifically, there are many topical products for use after exposure to the sun. These products are generally in the form of lotions or creams containing aloe vera and other moisturizers for hydrating and protecting the skin from the exposure. Other ingredients are also used to maintain the skin, sooth minor burns, and prevent peeling. Many people use these formulations to protect and preserve their skin when they have ordinary or excessive exposure to the sun. They can also provide an opportunity to help prevent skin cancer. According to the present invention, the addition of vitamin D₃ to such formulations provides for application of vitamin D₃ to the skin where it provides its cancer inhibiting effects.

The amount and form for adding vitamin D₃ to topical after sun lotions in order to best achieve the benefits is still subject to research and testing. However, synthetic or natural sources of vitamin D₃, such as cod-liver oil, can be used. The topical application of vitamin D is safe and beneficial. Schering-Plough's “A&D Ointment,” a preparation for diaper rash, has been available over the counter for decades. It contains approximately the adult recommended daily allowance of vitamin D (10 mcg or 400 units) per ounce. An ounce is approximately the amount necessary to cover an adult body. Various vitamins, herbs, and other ingredients are added to after sun lotions. Vitamin D₃ could also be added. Since vitamin D₃ is lipid soluble, the after sun lotion should contain lipids. Otherwise, any after sun formulation can be used with the present invention.

Because of its effects on growth and differentiation, vitamin D and its analogs are of potential benefit in the treatment of photoaging. Nagpal S, et al. Vitamin D analogs: mechanism of action and therapeutic applications. Curr Med Chem 2001; 8:1661-79. Thus, the addition of vitamin D₃ to after sun lotions may also inhibit photoaging.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. A method for inhibiting the formation and growth of cancerous and precancerous skin cells, the method comprising the steps of:
adding vitamin D₃ to a topical after-sun product; and
applying the after-sun product to at least portions of the skin exposed to sunlight.

2. The method of claim 1, wherein the vitamin D₃ is a formation of cholecalciferol.

3. The method of claim 1, wherein the vitamin D₃ includes cod liver oil.

4. The method of claim 1, wherein the after-sun product includes a lipid containing after-sun product.

5. The method of claim 1, wherein up to 5% of vitamin D₃ is added to the after-sun product.

6. The method of claim 1, wherein the after-sun product is a lotion.

7. The method of claim 1, wherein the after-sun product is a cream.

8. A method for reducing photoaging comprising the steps of:
adding vitamin D₃ to a topical after-sun product; and
applying the after-sun product to at least portions of the skin exposed to sunlight.

9. A formulation for inhibiting the formation and growth of cancerous and precancerous skin cells comprising:
a topical after-sun product; and
vitamin D₃.

10. The formulation according to claim 9, wherein the vitamin D₃ is a formation of cholecalciferol.

11. The formulation according to claim 9, wherein the vitamin D₃ includes cod liver oil.

12. The formulation according to claim 9, wherein the sunscreen includes a lipid containing after-sun product.

13. The formulation according to claim 9, wherein the vitamin D₃ is up to 5% of the after sun product.

14. The formulation according to claim 9, wherein the after-sun product is a lotion.

15. The formulation according to claim 9, wherein the after-sun product is a cream.
